(19) United States
(12) Patent Application Publication
(10) Pub. No.: US 2017/0226165 A1
(43) Pub. Date: Aug. 10, 2017

(54) SYSTEM AND METHOD FOR ISOLATION OF PROTEIN AS A CO-PRODUCT OF BIOFUEL AND/OR BIOCHEMICAL PRODUCTION

(71) Applicant: Fluid Quip Process Technologies, LLC, Springfield, OH (US)

(72) Inventors: Michael Franko, Denver, CO (US); Neal Jakel, Cedar Rapids, IA (US)

(21) Appl. No.: 15/427,541

(22) Filed: Feb. 8, 2017

Related U.S. Application Data
(60) Provisional application No. 62/293,107, filed on Feb. 9, 2016.

(51) Int. Cl. C07K 14/415; B02C 9/04; B01D 17/02; C07K 1/36

(52) U.S. Cl. CPC C07K 14/415; C07K 1/36; B02C 9/04; B01D 17/0217

(57) ABSTRACT

A method for protein isolation as a co-product of alcohol production is disclosed. The method includes receiving grain product at a holding receptacle, the grain product being at least one of whole stillage, dried distillers grain, and wet distillers grain. The method further includes directing the grain product from the holding receptacle over a plurality of screens, thereby allowing a fiber-containing portion to be collected on the screens and further allowing a protein-containing portion and an oil-containing portion to pass through the screens. The method further includes directing the protein-containing portion and the oil-containing portion optionally over a finishing screen, thereby allowing remaining fiber fractions to be collected on the finishing screen and further allowing the protein-containing and oil-containing portion to pass through the finishing screen. Further, the method includes centrifuging the protein-containing portion and the oil-containing portion for isolating a protein fraction and an oil, water, sugar and minerals fraction.

### FIELD OF THE INVENTION

The present invention generally relates to the field of biofuel and/or biochemical production, and more particularly to a system and method for isolation of protein as a co-product of alcohol (e.g., ethanol or butanol) production.

### BACKGROUND

Manufacturers use two major processes to produce alcohol or other starch-based products. These two processes, dry milling (mash distillation) and wet milling are very different, as are their co-products. The primary co-products from dry milling are distillers' grains, while the primary co-products from wet milling are gluten products. Co-products from the dry milling and/or wet milling of corn have important nutritional properties that add value to feeding rations and livestock programs. However, current systems and methods of isolating co-products from dry milling and wet milling processes may require unnecessary steps and equipment, which may be time-consuming and costly. Further, current systems and methods for isolating co-products may not efficiently utilize the various co-products.

Accordingly, it would be desirable to provide a system and method for isolating protein as a co-product of alcohol (e.g., ethanol or butanol) production that addresses the above-referenced shortcomings of current solutions.

### SUMMARY OF THE INVENTION

Accordingly, an embodiment of the present invention is directed to a method for protein isolation as a co-product of biofuel and/or biochemical production, including: receiving a grain product at a holding receptacle, the grain product being at least one of whole stillage, dried distillers grain and wet distillers grain; directing the grain product from the holding receptacle over a plurality of screens, thereby allowing a fiber-containing portion of the grain product to be collected on the plurality of screens and further allowing a protein-containing portion of the grain product and an oil-containing portion of the grain product to pass through the plurality of screens; directing the protein-containing portion and the oil-containing portion over a finishing screen, thereby allowing remaining fiber fractions from the protein-containing portion and the oil-containing portion to be collected on the finishing screen and further allowing the protein-containing portion and the oil-containing portion to pass through the finishing screen and centrifuging the protein-containing portion and the oil-containing portion for isolating a protein fraction and an oil, water, sugar, and minerals fraction; and recovering the isolated protein fraction.

An additional embodiment is directed to a system for protein isolation as a co-product of biofuel and/or biochemical production, including: a holding receptacle configured for receiving grain product, the grain product being at least one of whole stillage, dried distillers grain and wet distillers grain; a screen system, the screen system including a plurality of screens, the screen system being configured for receiving the grain product from the holding receptacle, each of the plurality of screens configured for retaining a fiber-containing portion of the grain product and further configured for allowing flow-through of a protein-containing portion of the grain product and an oil-containing portion of the grain product; a transfer receptacle, the transfer receptacle being configured for receiving the protein-containing portion of the grain product and the oil-containing portion of the grain product from the screen system; a finishing screen, the finishing screen being configured for receiving the protein-containing portion and the oil-retaining portion from the transfer receptacle, the finishing screen further being configured for retaining remaining fiber fractions of the protein-containing portion and the oil-containing portion, the finishing screen further being configured for allowing flow-through of the protein-containing portion and the oil-containing portion; and a centrifuge assembly, the centrifuge assembly being configured for receiving the protein-containing portion and the oil-retaining portion which pass through the finishing screen, the centrifuge assembly being further configured for centrifuging the protein-containing portion and the oil-containing portion for isolating a protein fraction and an oil, water, sugar, and minerals fraction.

It is to be understood that both the foregoing general description and the following detailed description are exemplary and explanatory only and are not necessarily restrictive of the invention as claimed. The accompanying drawings, which are incorporated in and constitute a part of the specification, illustrate embodiments of the invention and together with the general description, serve to explain the principles of the invention.

### BRIEF DESCRIPTION OF THE DRAWINGS

The numerous objects and advantages of the present invention may be better understood by those skilled in the art by reference to the accompanying figures in which:

FIGS. 1 and 3 are block diagram illustrations of exemplary systems for dry milling of corn;

FIG. 2 is a block diagram illustration of an exemplary system for wet milling of corn;

FIGS. 4A and 4B are block diagram illustrations of a system for isolation of protein as a co-product of alcohol production in accordance with an exemplary embodiment of the present invention;

FIGS. 4C and 4D are block diagram illustrations of a system for isolation of protein as a co-product of alcohol production in accordance with another exemplary embodiment of the present invention; and

FIG. 5 is a flowchart illustrating a method for protein isolation as a co-product of alcohol production.

### DESCRIPTION OF THE INVENTION

Reference will now be made in detail to the presently preferred embodiments of the invention, examples of which are illustrated in the accompanying drawings.

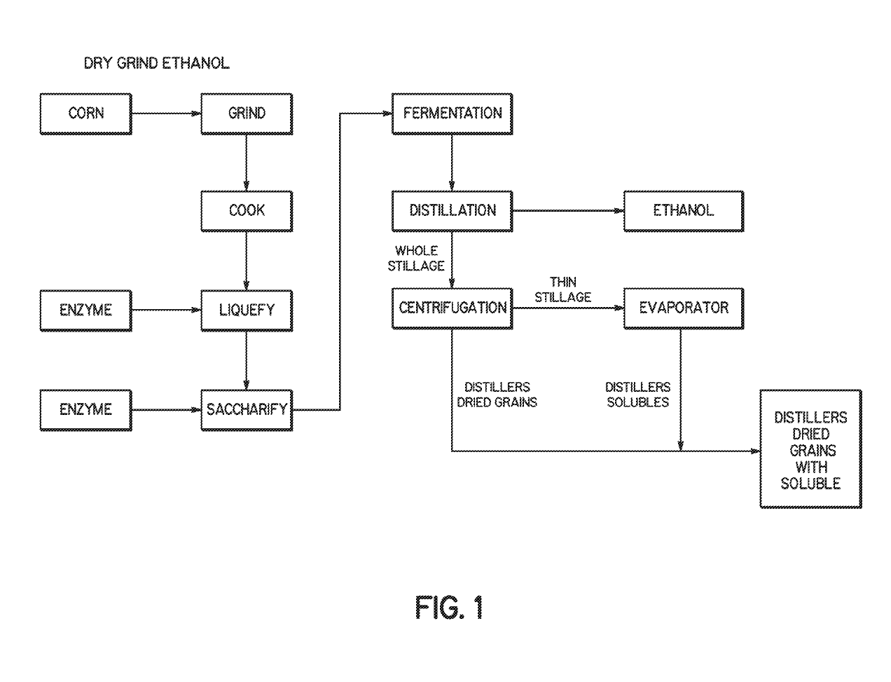

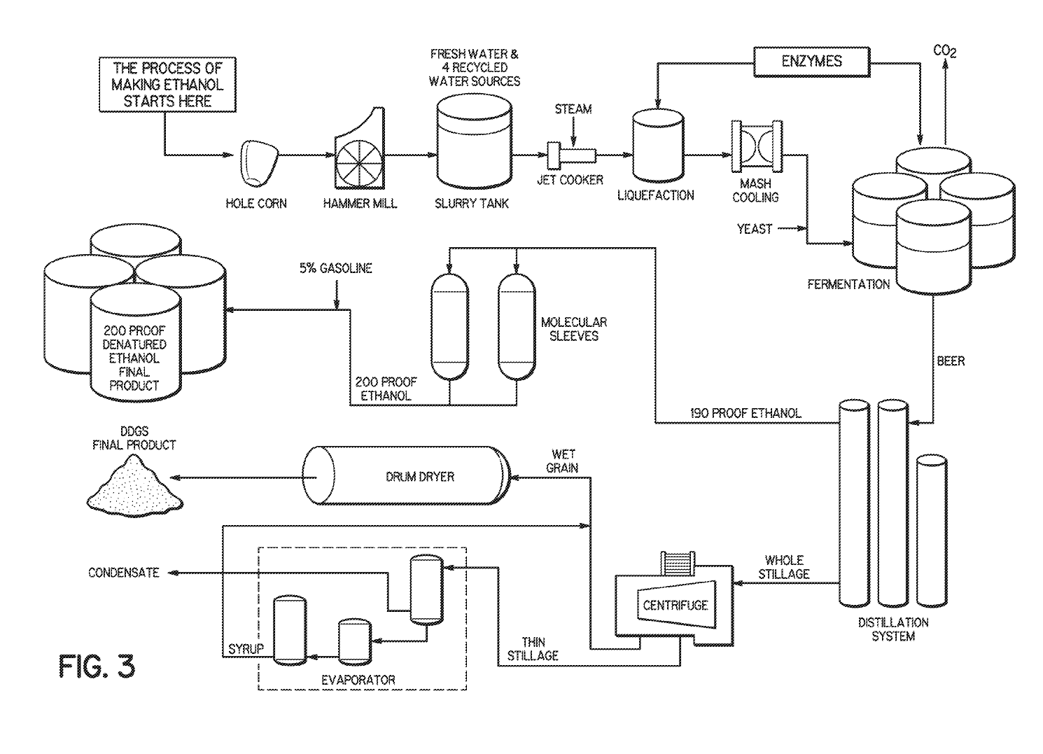

Referring generally to FIGS. 1 and 3, exemplary systems for dry milling of grain, such as corn, are shown. Typical dry milling systems, as shown, may produce ethanol (or other biofuels and/or biochemicals) via the following steps: corn is ground into coarse flour; the coarse flour is combined with water and/or enzymes are added to convert starch to sugar (thus forming the “mash”); the mash is cooked and sterilized; the mash is cooled; yeast/bacteria and enzymes are added to the mash to allow the sugar to convert to an organic compound, such as ethanol, with or without carbon dioxide (via fermentation); the mash is sent to distillation where the organic compound is separated leaving “spent mash” or whole stillage; the whole stillage goes to either screen/press or centrifuge (ex-decanting centrifuge) to separate out as much liquid as possible (the liquid being distillers solubles or “thin stillage”); the thin stillage may then go back into the system (i.e., be recycled), be sold as livestock feed, or be made into a syrup known as condensed distillers solubles (CDS); and the remaining part of the whole stillage (the wet grain/wet distillers grain) may be sold as livestock feed, dried into dried distillers grain (DDG), such as via a dryer, or dried into DDG to which syrup (CDS) may be added to produce distillers dried grains w/solubles (DDGS).

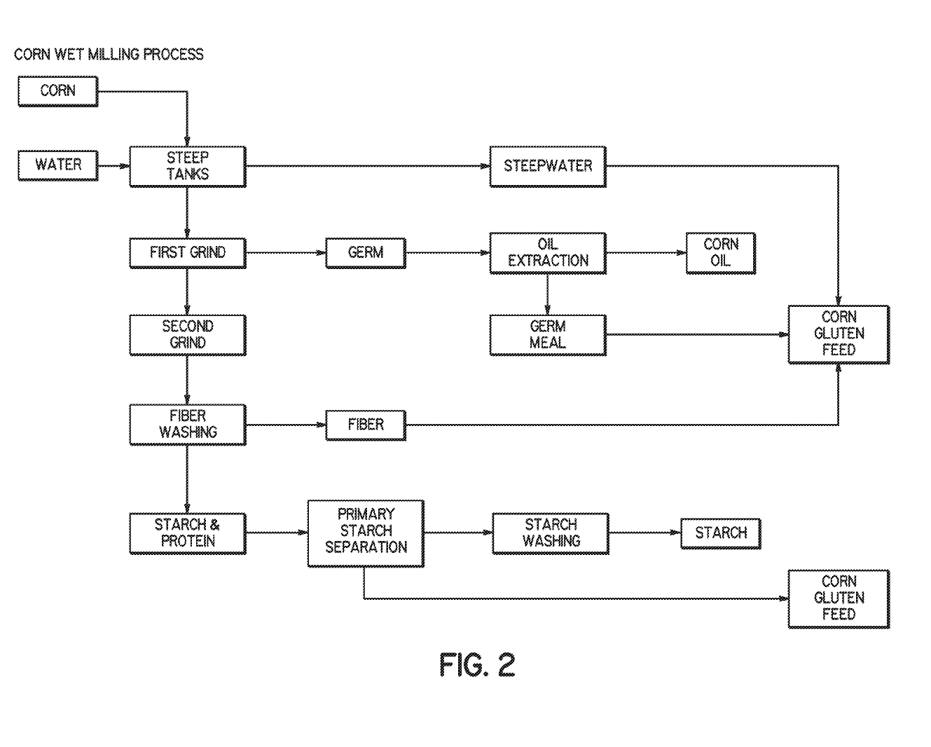

Referring generally to FIG. 2, an exemplary system for wet milling of corn is shown. A typical wet milling system produces alcohol or other starch-based products via the following steps: corn is soaked in a steep tank w/sulfur dioxide treated water to separate the starch and insoluble protein; the corn is removed and coarse milled (1st grind) to separate out the germ, wherein oil may be extracted from the germ and refined into corn oil, while the germ may be dried to form corn germ meal; the corn (w/o germ) is pulverized (2nd grind), washed and screened to separate the fiber (bran) from the starch and gluten/protein; the corn (w/o germ and fiber) is then centrifuged to separate the starch from the gluten/protein; the gluten/protein (60% protein) is concentrated, filtered and dried to produce corn gluten meal; the starch is separated again to reduce protein content to 0.3%, thereby producing purified starch, which may be used to make biofuels and/or biochemicals, such as ethanol and corn sweeteners.

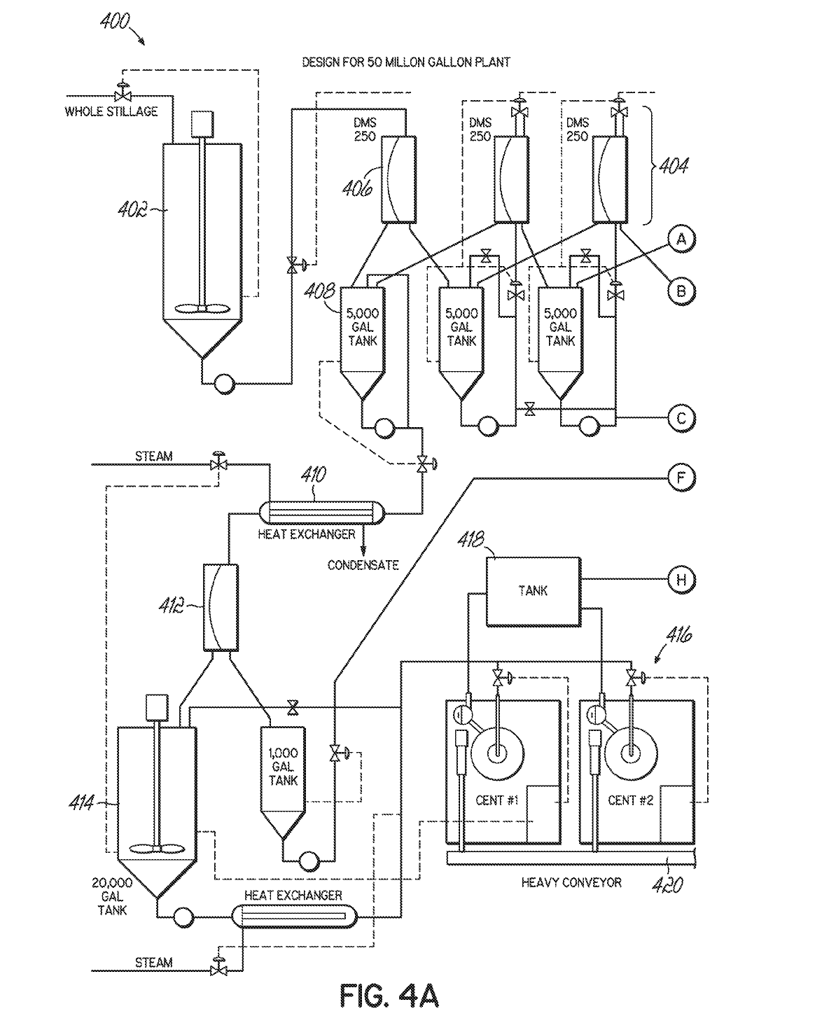

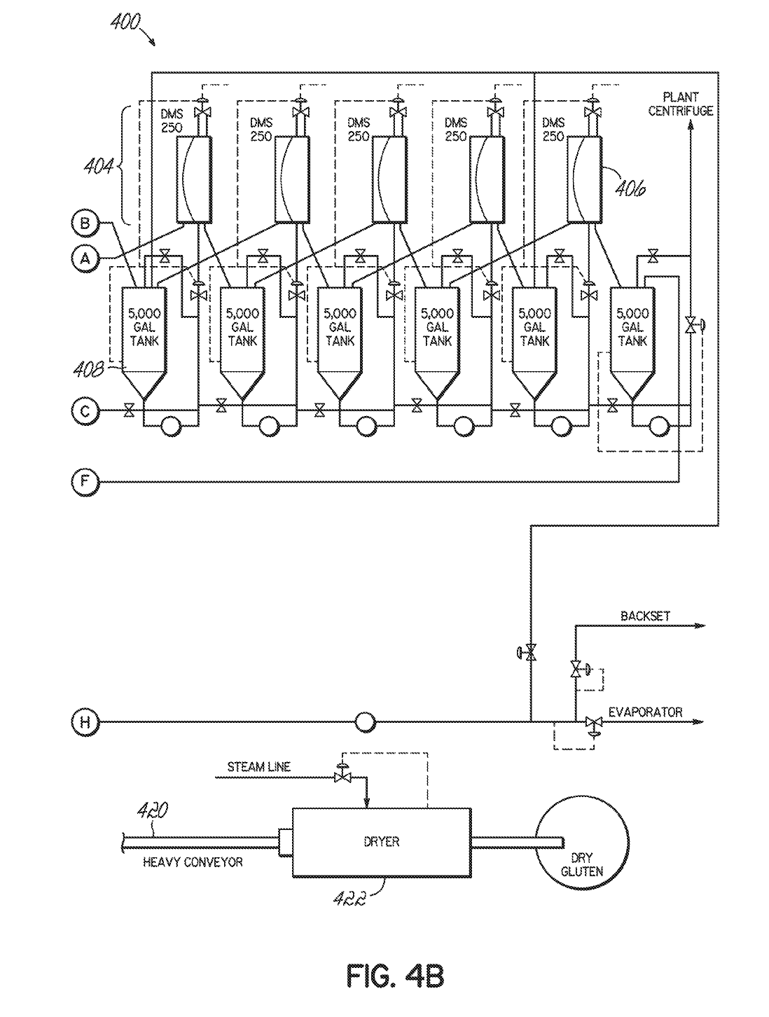

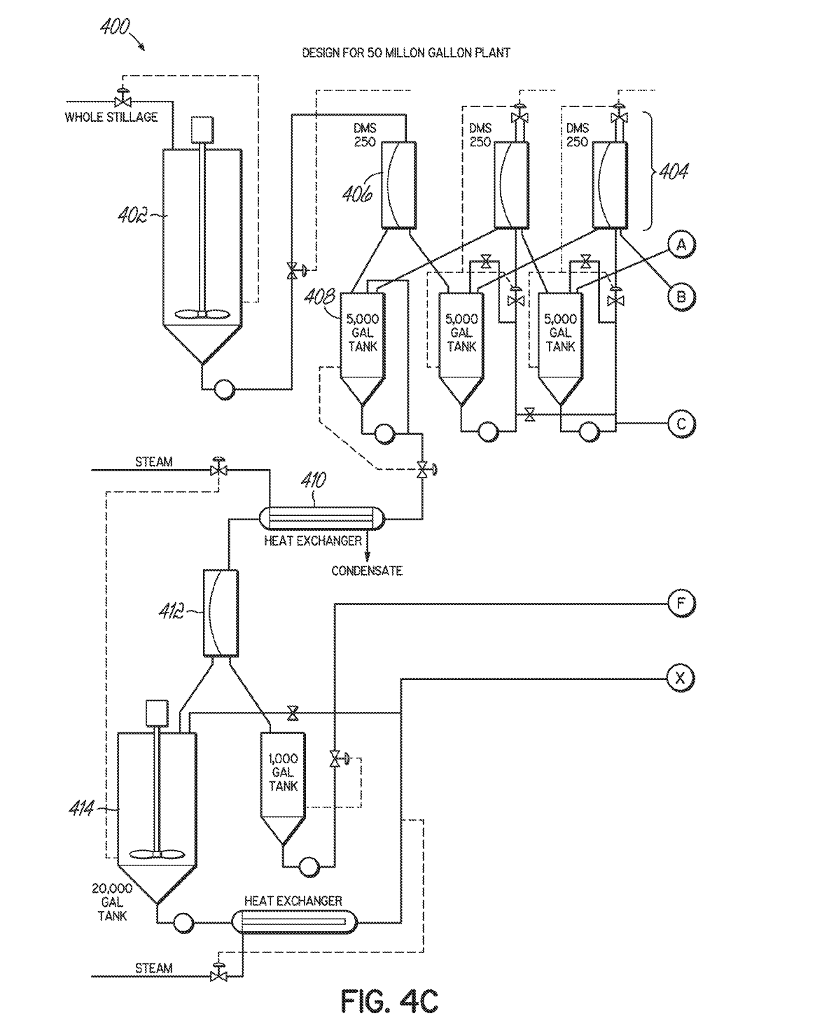

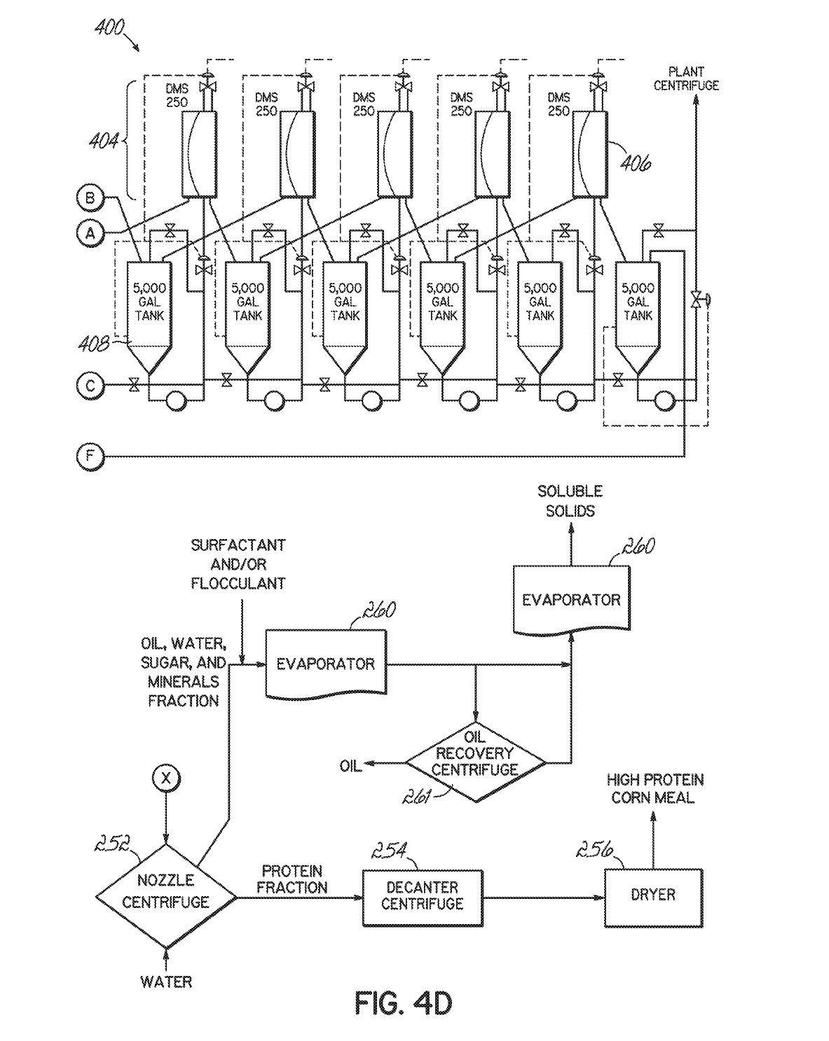

Referring generally to FIGS. 4A and 4B, a system for protein (ex-gluten) isolation as a co-product of biofuel (ex-ethanol) production in accordance with an exemplary embodiment of the present invention is shown. In a present embodiment, the system 400 may include a holding receptacle 402, such as a 40,000 gallon tank, which may be configured for receiving a grain product. The grain may be selected from corn, wheat, barley, oat, sugar cane, sorghum, and the like. In an exemplary embodiment, the grain product may be whole stillage, such as the post-distillation product (ex-spent mash) described above. In further embodiments, the grain product may be dried distillers grain (DDG) as described above. In still further embodiments, the grain product may be wet grain/wet distillers grain (which may be sixty (60) percent moisture) as described above. For example, the grain product may be received from an ethanol plant and may be a product/co-product that remains after completion of the distillation stage in a dry milling system as shown in FIGS. 1 and 3 (i.e., mash minus ethanol). In an exemplary embodiment, the grain product is directed from the holding receptacle 402 to a screen system 404. The screen system 404 may include a plurality or series of screens, such as gravity-fed or pressure screen 406 (e.g., a PS-Triple pressure screen available from Fluid-Quip of Springfield, Ohio), and may be configured for receiving the grain product from the holding receptacle 402. For instance, the screen system 404 may include eight pressure screens 406, each screen 406 being a two hundred and fifty (250) micron screen. In additional embodiments the screen may be any size included in the range of fifty (50) microns through four hundred (400) microns. In further embodiments, each screen 406 may be a one hundred and eighty degree screen.

In exemplary embodiments, the grain product may be pumped over the series of screens 406, with each of the plurality of screens 406 configured for retaining a fiber-containing portion of the grain product (i.e., the overflow or portion which passes over the screens 406). Further, each of the plurality of screens 406 is configured for allowing flow-through of a protein-containing portion of the grain product (i.e., the underflow) and an oil-containing portion of the grain product (i.e., the underflow). For example, approximately eighty percent (80%) of total available oil of the grain product may pass through the screens as underflow. The flow-through (ex- the oil-containing portion and the protein-containing portion) at each screen 406 may be captured by a series of transfer receptacles 408, from which, the flow-through may be directed/pumped over the next screen 406 of the screen system 404. For instance, the transfer receptacles may be a series of five thousand (5,000) gallon tanks. In current embodiments of the present invention, process water may be added to the overflow (ex- the fiber-containing portion) for hydrating the fiber overflow as may be necessary for facilitating pumping of the grain product (ex-fiber-containing portion) over the screens 406. This may be advantageous in that no fresh water may need to be added to the system 400.

In additional embodiments, the grain product may be fed to/pumped to/pumped over the screens 406 at a header pressure, for example, of at least sixty (60) pounds per square inch (psi). In another example, the grain product may be fed to/pumped to/pumped over the screens 406 at a header pressure of between one hundred (100) psi and one hundred and twenty (120) psi. Further, as the grain product is pumped through/over the series of screens, it may be maintained at a temperature of at least or approximately one hundred eighty (180) degrees Fahrenheit. In current embodiments of the present invention, one or more heat exchangers 410 may be included/connected in-line for promoting maintenance of the temperature of the grain product at the desired temperature (ex-one hundred eighty (180) degrees Fahrenheit up to about two hundred forty (240) degrees Fahrenheit). Also, as the grain product is pumped over/through the series of screens 406, a continuous flow counter current may be created meaning that the flow-through (ex-protein containing portion and oil-containing portion) moves backward (ex-to a preceding transfer receptacle 408), while the overflow (ex-fiber-containing portion) moves forward (to a next screen 406) (as shown in FIGS. 4A and 4B).

In exemplary embodiments, once the grain product and/or fiber-containing portion/overflow has been pumped over all of the screens 406 of the screen system 404, the fiber-containing portion which had been collected on and pumped over the screens 406 may be directed to/pumped back to the ethanol plant. For example, once at the ethanol plant, the fiber-containing portion may be: a) placed in a decanting centrifuge and dried; b) sold as a wet product or c) further processed for conversion to sugars for biofuel and/or biochemical production. The feed value of the fiber-containing portion/overflow/feed stock/feed product may be approximately 20% protein, 15% fiber and 5% fat.

In further embodiments, the system 400 may include a finishing screen 412. The finishing screen 412 may be configured for receiving the protein-containing portion and the oil-containing portion from one of the transfer receptacles 408. Further, the finishing screen 412 may be a gravity-fed or pressure screen (e.g., a PS-Triple pressure screen available from Fluid-Quip of Springfield, Ohio). In exemplary embodiments, the finishing screen 412 is further configured for retaining remaining fiber fractions of the protein-containing portion and the oil-containing portion which were not retained/captured by the screens 406 of the screen system 404. The finishing screen 412 is also configured for allowing flow-through of the protein-containing portion of the grain product and the oil-containing portion of the grain product. For promoting its fiber fraction retention functionality, the finishing screen 412 may have a smaller slot width than the screens 406 of the screen system 404. For example, the finishing screen 412 may be a seventy-five (75) micron screen, but may also be any size included in the range of fifty (50) microns through two hundred fifty (250) microns. In additional embodiments, the finishing screen 412 may be a one hundred and eighty (180) degree screen. Further, one or more of the heat exchangers 410 may be included/connected in-line for promoting maintenance of the temperature of the protein-containing portion and/or the oil-containing portion at the desired temperature (ex-one hundred eighty (180) degrees Fahrenheit), or for increasing temperature. The protein-containing portion and the oil-containing portion, which flow-through the finishing screen, may be received in a receiving vessel or tank 414.

In additional embodiments, the system 400 includes a centrifuge assembly 416. The centrifuge assembly 416 may be a 2-stage centrifuge assembly configured for receiving the protein-containing portion and the oil-containing portion which pass through the finishing screen 412. In exemplary embodiments, the protein-containing portion and the oil-containing portion may be directed or pumped from the receiving vessel/tank 414 to the centrifuge assembly 416. The centrifuge assembly 416 may be further configured for centrifuging the protein-containing portion and the oil-containing portion for isolating a protein fraction and an oil, water, sugar, and minerals fraction. In current embodiments of the present invention, the isolated oil, water, sugar, and minerals fraction may pass from/exit the centrifuge assembly 416 as centrifuge overflow.

The oil, water, sugar and minerals fraction may be directed/pumped to the ethanol plant where it may be used as backset, used to hydrate new grain/corn before processing and/or evaporated via an evaporator of the ethanol plant. The evaporator can concentrate the fraction to about 35% solids, or higher or lower. The concentrated stream can be further processed to separate the oil from the water, sugar, and minerals to produce a pure oil fraction of 96% or greater concentration. Still further, the oil, water, sugar, and minerals fraction may be recycled/pumped back through the system 400 and used as process water, which may be used to hydrate the fiber overflow as discussed above. Surfactant and/or flocculant may be added prior to or during evaporation and before oil separation to improve oil separation efficiency and/or yield.

In further embodiments of the present invention, the isolated protein fraction may pass from/exit the centrifuge assembly 416 as centrifuge underflow. For example, the protein fraction may be approximately 75% moisture, approximately 40-55% protein, approximately 20% dry solids, approximately 5% fiber, and approximately 3% fat. Because free oil, water, sugar, and minerals have been removed from the protein fraction via centrifugation, the protein fraction may be a “purified” protein stream and may contain yeast and yeast protein. In additional embodiments, the system 400 may include a conveyer 420, such as a heavy conveyer, for transporting the protein fraction from the centrifuge assembly 416 to a dryer 422. For instance, the dryer 422 may be a rotary, ring or drum dryer. In exemplary embodiments, the dryer 422 may be configured for drying/removing moisture from the isolated protein fraction. For example, the isolated protein fraction may be dried to approximately 10% moisture and stockpiled for shipping as a high feed value product. Further processing of the protein fraction may include the separation of specific amino acids, the separation of zein protein and/or the separation of the yeast components within the protein fraction by processes known in the art. The isolated protein fraction may also be utilized as a feed source to produce corn protein concentrates or isolates.

Referring now to FIGS. 4C and 4D, in one example, the centrifuge assembly 416 can include a nozzle centrifuge 252 that is configured for receiving the protein-containing portion and the oil-containing portion. The nozzle centrifuge 252 can be provided with washing capabilities so that water, along with the protein-containing portion and the oil-containing portion, can be supplied to the nozzle centrifuge 252. The additional water allows for easier separation into a protein fraction and an oil, water, sugar and minerals fraction. The heavier protein fraction separates from the oil, water, sugar and minerals fraction and is removed as the underflow, whereas the lighter oil, water, sugar, and minerals fraction can be removed as the overflow. One such suitable nozzle centrifuge 252 is the FQC-950 available from Fluid-Quip, Inc. of Springfield, Ohio. In an alternate embodiment, the nozzle centrifuge 252 can be replaced with a standard cyclone apparatus or other like device, as are known in the art, to separate the protein-containing portion and the oil-containing portion into the underflow protein fraction and overflow oil, water, sugar and minerals fraction. One such suitable cyclone apparatus is the RM-12-688 available from Fluid-Quip, Inc. of Springfield, Ohio. The oil, water, sugar and minerals fraction may be directed/pumped to the ethanol plant where it may be used as backset, used to hydrate new grain/corn before processing and/or evaporated via an evaporator of the ethanol plant. Still further, the oil, water, sugar and minerals fraction may be recycled/pumped back through the system 400 and used as process water, which may be used to hydrate the fiber overflow as discussed above.

With further reference now to FIG. 4D, the underflow protein fraction from the nozzle centrifuge 252 is shown as being further piped and subjected to decanter centrifuge 254 to dewater the protein fraction. The decanter centrifuge 254 is standard and known in the art. One such suitable decanter centrifuge 254 is the NX-944HS available from Alfa Laval of Lund, Sweden. Other like devices are contemplated. The separated water portion or filtrate from the decanter centrifuge 254 may be recycled back, for example, to the liquefaction step or the fermentation step for reuse in the dry mill process (FIGS. 1 and 3). The dewatered protein fraction is then dried, such as by being sent to a dryer 256, as is known in the art. In an alternate embodiment, the dewatered protein portion can be subjected to vacuum filtration or other drying methods, as are known in the art. The final dried protein fraction defines a high protein corn meal that includes at least 40 wt % protein on a dry basis and which may be sold as pig or chicken feed, for example. In another embodiment, the high protein corn meal includes at least 45 wt % protein on a dry basis. In another embodiment, the high protein corn meal includes at least 50 wt % protein on a dry basis. In yet another embodiment, the high protein corn meal includes at least 60 wt % protein on a dry basis. In still another embodiment, the high protein corn meal includes about 56 wt % protein on a dry basis. The resulting high protein corn meal may be sold at a much higher cost per ton than distillers dry grains with solubles (DDGS) or distillers wet grains with solubles (DWGS).

With continuing reference to FIGS. 4C and 4D, the overflow oil, water, sugar, and minerals fraction is piped from the nozzle centrifuge 252 and subjected to an evaporator(s) 260, as are known in the art, to begin separating the soluble solids from the water soluble solids portion of the oil, water, sugar and minerals fraction. The evaporator(s) 260 evaporates the liquid portion of the oil, water, sugar and minerals fraction. Thereafter, the remaining oil, water, sugar, and minerals fraction can be piped and subjected to an optional oil recovery centrifuge 261, as is known in the art, so that the oil can be removed therefrom. One such suitable oil recovery centrifuge 261 is the ORPX 617 available from Alfa Laval of Lund, Sweden. In one example, the final recovered oil product can include between about 40 wt % to about 60 wt % of the total corn oil in the corn. In comparison to typical oil recovery in a standard dry mill process, oil recovery centrifuge 261 can function at a higher capacity because the water soluble solids portion, which is subjected to the oil recovery centrifuge 261, includes less liquid, less protein and less fiber than normal.

The remainder of the oil, water, sugar and minerals fraction can be piped and subjected to an additional evaporator(s) 260 whereat the liquid portion is further evaporated to ultimately yield a soluble solids portion. While the oil, water, sugar and minerals fraction is subjected to at least two evaporators 260, it should be understood that the number of evaporators or sets thereof can be varied, i.e., can be more or less, from that shown depending on the particular application and result desired.

The resulting soluble solids portion may be combined with the fiber-containing portion or overflow which had been collected on and pumped over the screens 406 to provide distillers wet grains with solubles (DWGS), which may be further dried by a drier, as is known in the art, to provide distillers dry grains with solubles (DDGS), both of which can be sold to dairy and beef feedlots. In another example, the soluble solids portion may be used as a natural fertilizer.

Accordingly, in this dry mill process, neither the DDGS nor DWGS receive the typical concentrated syrup from the evaporators 260. While this change from the typical dry mill process results in a lower crude protein content in the DDGS and DWGS, this decrease is insubstantial, particularly when the economic advantages of producing the high protein corn meal are realized. And, despite the lower protein content, the DDGS and DWGS may still be sold to beef and dairy feedlots as cattle feed.

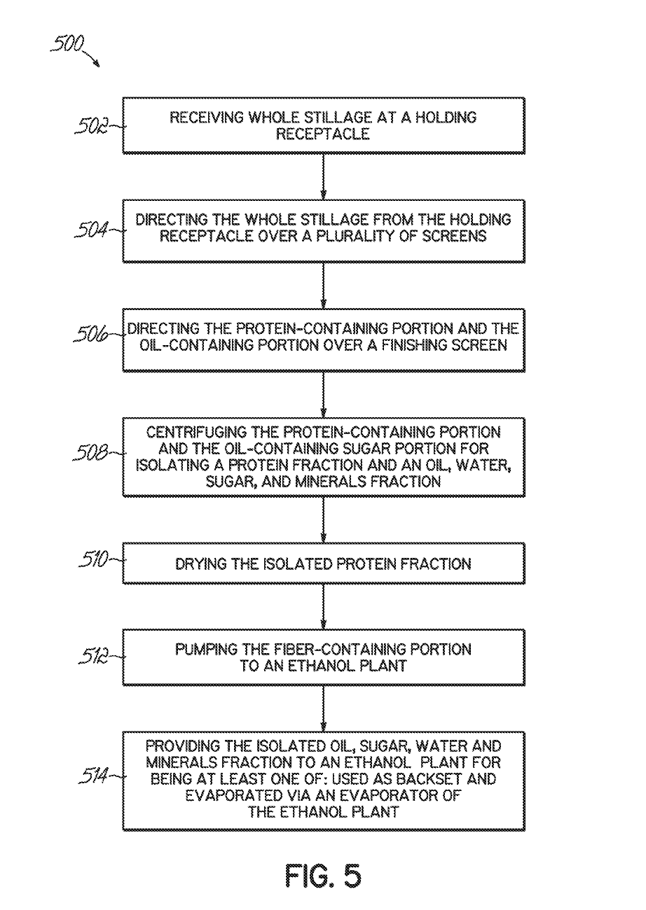

Referring generally to FIG. 5, a flowchart illustrating a method for protein isolation as a co-product of alcohol production is shown. In an exemplary embodiment, the method 500 includes receiving grain product at a holding receptacle 502. The method 500 may further include directing the grain product from the holding receptacle over a plurality of screens 504, thereby allowing a fiber-containing portion of the grain product to be collected on the plurality of screens and further allowing a protein-containing portion of the grain product and an oil-containing portion of the grain product to pass through the plurality of screens. In exemplary embodiments, the fiber-containing portion includes between 10-30% protein, between 5-25% fiber and between 1-10% fat. The method 500 may further include directing the protein-containing portion and the oil-containing portion over a finishing screen 506, thereby allowing remaining fiber fractions from the protein-containing portion and the oil-containing portion to be collected on the finishing screen and further allowing the protein-containing portion and the oil-containing portion to pass through the finishing screen. The method 500 may further include centrifuging the protein-containing portion and the oil-containing portion for isolating a protein fraction and an oil, water, sugar and minerals fraction 508. In current embodiments of the present invention, the isolated oil fraction includes at least ninety-six percent (96%) pure corn oil. In exemplary embodiments, the isolated protein fraction may include between thirty percent (30%) and sixty-five percent (65%) protein. In further embodiments, the isolated protein fraction may include between ten percent (10%) and thirty percent (30%) dry solids. In still further embodiments, the isolated protein fraction may include between seventy percent (70%) and ninety-five percent (95%) moisture.

In additional embodiments, the method 500 may further include drying the isolated protein fraction 510. In exemplary embodiments, the dried isolated protein fraction includes between five percent (5%) and fifteen (15%) moisture. The method 500 may further include pumping the fiber-containing portion to an ethanol plant 512. The method 500 may further include providing the isolated water and minerals fraction to an ethanol plant for being at least one of: used as backset and evaporated via an evaporator of the ethanol plant 514.

In further embodiments, the method 500 may include adding process water for hydrating the fiber-containing portion collected on the plurality of screens 516. In exemplary embodiments, at least one of the grain product, the protein-containing portion and the oil-containing portion is maintained at a temperature of at least one hundred eighty (180) degrees Fahrenheit. In additional embodiments, the step of directing the grain product over the plurality of screens 504 and/or the step of directing the protein-containing portion and the oil-containing portion over a finishing screen 506 includes pumping the grain product to the plurality of screens at a header pressure of at least sixty (60) pounds per square inch (psi), but preferably between one hundred (100) pounds per square inch (psi) and one hundred twenty (120) pounds per square inch (psi).

It is noted that in embodiments in which the grain product is wet distillers grain or dried distillers grain, water may need to be added during the process to facilitate pumping of the wet distillers grain or dried distillers grain through the system 400.

It is believed that the system and method for isolation of protein as a co-product of biofuel and/or biochemical production of the present invention and many of its attendant advantages will be understood by the forgoing description. It is also believed that it will be apparent that various changes may be made in the form, construction and arrangement of the components and steps thereof without departing from the scope and spirit of the invention or without sacrificing all of its material advantages. The form herein before described being merely an explanatory embodiment thereof.

What is claimed is:

1. A method for protein isolation as a co-product of biofuel and/or biochemical production, comprising:
receiving a grain product at a holding receptacle, the grain product being at least one of whole stillage, dried distillers grain and wet distillers grain;
directing the grain product from the holding receptacle over a plurality of screens, thereby allowing a fiber-containing portion of the grain product to be collected on the plurality of screens and further allowing a protein-containing portion of the grain product and an oil-containing portion of the grain product to pass through the plurality of screens;
directing the protein-containing portion and the oil-containing portion over a finishing screen, thereby allowing remaining fiber fractions from the protein-containing portion and the oil-containing portion to be collected on the finishing screen and further allowing the protein-containing portion and the oil-containing portion to pass through the finishing screen;
centrifuging the protein-containing portion and the oil-containing portion to isolate a protein fraction and an oil, water, sugar and minerals fraction; and
recovering the isolated protein fraction.

2. The method as claimed in claim 1, further comprising drying the isolated protein fraction.

3. The method as claimed in claim 1, wherein the isolated oil fraction includes at least ninety-nine percent (99%) pure corn oil.

4. The method as claimed in claim 1, wherein the isolated protein fraction includes between thirty percent (30%) and sixty-five percent (65%) protein.

5. The method as claimed in claim 1, wherein the isolated protein fraction includes between ten percent (10%) and thirty percent (30%) dry solids.

6. The method as claimed in claim 1, wherein the isolated protein fraction includes between seventy percent (70%) and ninety percent (90%) moisture.

7. The method as claimed in claim 1, wherein the fiber-containing portion includes between 10-30% protein, between 5-25% fiber and between 1-10% fat.

8. The method as claimed in claim 2, wherein the dried isolated protein fraction includes between five percent (5%) and fifteen (15%) moisture.

9. The method as claimed in claim 1, wherein the step of directing the grain product over the plurality of screens includes pumping the grain product to the plurality of screens at a header pressure of at least one hundred (100) pounds per square inch (psi).

10. The method as claimed in claim 1, wherein at least one of the grain product, the protein-containing portion and the oil-containing portion is maintained at a temperature of at least one hundred eighty (180) degrees Fahrenheit.

11. The method as claimed in claim 1, further comprising dewatering the isolated protein fraction.

12. A system for protein isolation as a co-product of biofuel and/or biochemical production, comprising:
a holding receptacle configured to receive a grain product, the grain product being at least one of whole stillage, dried distillers grain and wet distillers grain;
a screen system, the screen system including a plurality of screens, the screen system being configured to receive the grain product from the holding receptacle, each of the plurality of screens configured to retain a fiber-containing portion of the grain product and further configured to allow flow-through of a protein-containing portion of the grain product and an oil-containing portion of the grain product;
a transfer receptacle, the transfer receptacle being configured to receive the protein-containing portion of the grain product and the oil-containing portion of the grain product from the screen system;
an optional finishing screen, the finishing screen being configured to receive the protein-containing portion and the oil-retaining portion from the transfer receptacle, the finishing screen further being configured to retain remaining fiber fractions of the protein-containing portion and the oil-containing portion, the finishing screen further being configured to allow flow-through of the protein-containing portion and the oil-containing portion; and
a centrifuge assembly, the centrifuge assembly being configured to receive the protein-containing portion and the oil-retaining portion which pass through the finishing screen, the centrifuge assembly being further configured to centrifuge the protein-containing portion and the oil-containing portion to isolate a protein fraction and oil, water, sugar, and minerals fraction.

13. The system as claimed in claim 12, further comprising a drum or ring dryer, the drum or ring dryer being configured to receive the isolated protein fraction from the centrifuge assembly, the drum or ring dryer being further configured to dry the isolated protein fraction.

14. The system as claimed in claim 12, wherein the finishing screen and the plurality of screens of the screen system are gravity-fed screens.

15. The system as claimed in claim 12, further comprising an inline heat exchanger configured to maintain a temperature of at least one of the grain product, the protein-containing portion and the oil-containing portion.

16. The system as claimed in claim 12, wherein each of the plurality of screens of the screen system are 50 to 400 micron screens.

17. The system as claimed in claim 12, wherein each of the plurality of screens of the screen system are 250 micron screens.

18. The system as claimed in claim 12, wherein the finishing screen is a 50 to 250 micron screen.

19. The system as claimed in claim 12, wherein the finishing screen is a 150 micron screen.

20. The system as claimed in claim 12, further comprising a dewatering apparatus configured to receive the isolated protein fraction from the centrifuge assembly and dewater the isolated protein fraction.
